(19) United States
(12) Patent Application Publication
(10) Pub. No.: US 2003/0139362 A1
(43) Pub. Date: Jul. 24, 2003

(54) Methods of treating cognitive decline disease conditions with androgens

(76) Inventors: Jacob Raber, Portland, OR (US); Lennart Mucke, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/256,676

(22) Filed: Sep. 27, 2002

Related U.S. Application Data
(63) Continuation-in-part of application No. PCT/US01/10448, filed on Mar. 29, 2001.
(60) Provisional application No. 60/193,005, filed on Mar. 29, 2000.

(51) Int. Cl. A61K 48/00; A61K 31/57

(52) U.S. Cl. 514/44; 514/177

(57) ABSTRACT

Methods for treating a host, particularly a female host, for a cognitive decline disease condition are provided. Also provided are methods for improving the cognitive function of a host. In the subject methods, an effective amount of androgenic agent, e.g., a male sex hormone such as testosterone or analogue thereof, is administered to the host, resulting in at least an improvement in cognitive function of the host. The subject methods find use in a variety of different applications, and are particularly suited for use in treating female hosts for neurodegenerative disease conditions, e.g., Alzheimer's disease. Also provided are kits for use in practicing the subject methods.

### CROSS-REFERENCE

This application is a continuation-in-part of International Patent Application No. PCT/US01/10448, filed Mar. 29, 2001, which is incorporated herein by reference in its entirety and to which application we claim priority under 35 USC § 120, and claims the benefit of U.S. Provisional Patent Application No. 60/193,005, filed Mar. 29, 2000, which application is incorporated herein by reference in its entirety.

### STATEMENT REGARDING FEDERALLY SPONSORED RESEARCH

The U.S. government may have certain rights in this invention, pursuant to grant nos. RO1-AG20904-01 awarded by the National Institutes of Health.

### FIELD OF THE INVENTION

The field of this invention is cognition and methods for enhancing cognitive function, e.g., for treatment of cognitive decline disease conditions, such as neurodegenerative disease conditions.

### BACKGROUND

Alzheimer's Disease (AD) is a progressive, neurodegenerative disease characterized by cognitive and non-cognitive behavioral changes which include: (a) memory loss; (b) language deterioration; (c) impaired visuo-spatial skills; (d) poor judgment; (e) indifferent attitude and (f) aimless, unpredictable behavior. Although AD usually begins after age 60, its onset may occur decades earlier. AD first appears as memory decline. As the disease progresses over several years, cognition, personality, and the ability to function are all impaired or destroyed. Confusion, anxiety, disruption of circadian rhythm, and restlessness may also occur. The type, severity, sequence, and progression of mental changes vary widely among AD patients. Some people have the disease for only the last 5 years of life, while others may have it for as many as 20 years.

There is no known cure for AD and no way to slow the progression of the disease. For some people in the early or middle stages of the disease, medication such as tacrine may alleviate some cognitive symptoms. Also, some medications may help control non-cognitive behavioral symptoms such as sleeplessness, agitation, wandering, anxiety, and depression. These treatments are aimed solely at making the patient more comfortable and do nothing to slow the progression of the underlying disease.

Increasingly, studies are reporting that estrogen may protect against the development of Alzheimer's disease. A number of studies have reported that women taking hormone replacement therapy (in various combinations) score better on memory and learning than women not on HRT and have slower decline in mental functioning. Clinical trials are being conducted in patients with AD to evaluate the benefit of estrogen in females and of testosterone in males. While estrogen treatment seems unable to slow disease progression (propagation phase), some studies suggest that it may delay or prevent the onset of AD (initiation phase(Yaffe et al. (2000) Neurology 54:1949-1954). Two studies found that women who took HRT had a reduced risk for Alzheimer's disease—in one the risk was lower by 60%. These reports are backed up by studies indicating that estrogen stimulates blood flow in the brain, increases production of a beneficial form of beta amyloid, and triggers the temporary growth of nerve pathways in the memory portion of the brain. However, estrogen had no beneficial effect on cognitive function in healthy elderly women that carry the APOE ε4 allele. It has been reported that the apoE4 isoform is associated with AD.

As is evident from the above discussion, there is a continued need for the development of new treatment modalities for individuals suffering from Alzheimer's disease and other neurodegenerative disorders.

Literature

U.S. patents of interest include: U.S. Pat. Nos. 5,508,167; 5,554,601; 5,716,828; 5,767,248; 5,776,923; 5,780,587; 5,795,883; 5,840,540; 5,889,042; 5,986,054; 5,935,781; 6,027,896; and 6,027,899. Also of interest are U.S. Pat. No. 6,175,057 and Raber et al., Proc. Nat'l Acad. Sci. USA (1998) 95: 10914-10919; and Farrer et al. (1997) J. Am. Med. Assoc. 278:1349-1356.

### SUMMARY OF THE INVENTION

Methods for treating a host, particularly a female host, for a cognitive decline disease condition are provided. Also provided are methods for improving the cognitive function of a host. In the subject methods, an effective amount of an androgenic agent, e.g., a male sex hormone such as testosterone or analogue thereof, is administered to the host, resulting in at least an improvement in cognitive function of the host. The subject methods find use in a variety of different applications, and are particularly suited for use in treating female hosts for neurodegenerative disease conditions, e.g., Alzheimer's disease. Also provided are kits for use in practicing the subject methods.

### BRIEF DESCRIPTION OF THE FIGURES

FIG. 1 provides a graphical depiction of the results observed using the water maze assay described in the Experimental section, infra.

FIG. 2 depicts water maze learning curves of tfm female and male mice.

FIG. 3 depicts probe trial performance of tfm female and male mice.

FIG. 4 depicts novel location recognition of tfm female and male mice.

FIGS. 5A-D depict cytosolic levels of AR in the neocortex and response to androgen treatment.

### DESCRIPTION OF THE SPECIFIC EMBODIMENTS

Methods for treating a host, particularly a female host, for a cognitive decline disease condition are provided. Also provided are methods for improving the cognitive function of a host. In the subject methods, an effective amount of androgenic agent, e.g., a male sex hormone such as testosterone or analogue thereof, is administered to the host, resulting in at least an improvement in cognitive function of the host. The subject methods find use in a variety of different applications, and are particularly suited for use in treating female hosts for neurodegenerative disease conditions, e.g., Alzheimer's disease. Also provided are kits for use in practicing the subject methods.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Treatment Methods

In the broadest sense, methods are provided for improving a cognitive function in a mammalian host. In practicing the subject methods, an effective amount of an agent that increases a level and/or an activity of an androgen receptor in the cytosol and/or nucleus is administered to the host to obtain the desired improvement in cognitive function. The host is generally a mammalian host, and in many embodiments is a human. In certain preferred embodiments, the host is a female host, e.g., a female human. In certain preferred embodiments, the host comprises at least one ApoE4 allele, such that it expresses the ApoE4 isoform and includes the ApoE4 isoform. As such, the host may be heterozygous or homozygous for the ApoE4 isoform. In certain preferred embodiments, the host is homozygous for ApoE4. In these embodiments, the host may be a male host that carries the ApoE4 allele, but in many embodiments is a female host.

The instant methods generally involve administering an agent that increases the level and/or the activity of an androgen receptor in the cytosol and/or nucleus of a cell. Suitable agents include androgenic agents; agents that increase the level of mRNA encoding an androgen receptor (AR); agents that decrease the binding of apoE4 to AR; agents that increase the level or activity of AR in the cytosol and/or nucleus; and the like.

Administration of an agent that increases a level and/or an activity of an androgen receptor to the host according to the subject methods results in an improvement in at least one cognitive function of the host. Cognitive functions of interest include: spatial and non-spatial learning and memory; and the like. In certain situations, treatment according to the subject methods results in a complete removal of a deficit in the cognitive function. The amount of improvement is at least about 2 fold, usually at least about 5 fold and more usually at least about 10 fold as compared to a suitable control, e.g., an otherwise substantially identical host not administered an androgenic agent, e.g., a female host having similar level of cognitive ability (e.g., suffering from the same cognitive decline disease condition such as Alzheimer's disease, etc.) that has been administered a placebo, where in certain embodiments the amount of improvement is at least about 25 fold, 50 fold, 75 fold, 100 fold or greater. In some situations, an individual being treated is not able to perform a certain test or function, which inability is indicative of reduced cognitive function, and treatment of such an individual using the instant methods results in the ability of an individual to perform the test or function. The cognitive function improvement can be evaluated using any convenient protocol, where suitable protocols include, but are not limited to: 1) Wechsler Adult Intelligence Scale(WAIS-R) (Wechsler, D. WAIS-R Manual. New York: Psychological Corporation, 1981; Ivnik et al. Mayo's older American normative studies:WAIS-R norms for age 56 to 97. Clin Neuropsychol 1992, 6 (suppl):1-30); 2) Mini-Mental State Examination (MMSE) (Folstein et al. Mini Mental State: a practical method for grading the cognitive state of patients for the clinician. J Psychiat Res 1975;12:189-98; Commenges et al. Statistical description of the Mini-Mental State Examination (MMS) for French elderly community residents. J Nerv Ment Diss 1992; 180:28-32); 3) Information-Memory-Concentration test (Blessed et al. The association between quatitative measures of dementia and of senile changes in the cerebral grey matter of elderly subjects. Br J Psychiat 1968;114:796-811); 4) Fuld Object Memory Evaluation (FOSE) (Fuld, Pa. The Fuld Object Memory Test. Chicago: The Stoeltimg Instrument Company, 1981); 5) The Buschke Selective Reminding Test (BSRT) (Buschke, H. Selective reminding for analysis of memory and learning. J Verbal Learn Verb Behav 1973; 12:543-50); 6) The Rey Auditory Recall Test (Buschke, H. Selective reminding for analysis of memory and learning. J Verbal Learn Verb Behav 1973;12:543-50); 7) The CERAD Word List (Morris et al. The consortium to establish a registry for Alzheimer's Disease (CERAD). Part I. Clinical and neuropsychological assessment of Alzheimer's Disease. Neurology 1989;39: 1159-65); 8) The Beton Visual Retention Test (BVRT) (Benton, Ala. The revised visual attention test, 4^{th} edn. New York: Psychological Corporation, 1974); 9) The California Verbal Learning Test (Delis et al. The California Verbal Learning Test. New York: Psychological Corporation, 1987); and 10) Assessment of navigation in humans (Maguire et al. Knowing where and getting there; a human navigation network, Science 1998; 280:921-924).

Androgenic Agents

In some embodiments, the methods involve administering an effective amount of androgenic agent to the host. Suitable androgenic agents that may be used in the subject methods include, but are not limited to: the naturally occurring androgens and derivatives thereof including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, dihydrotestosterone (DHT), biologically active analogs of DHT, 4-dihydrotestosterone (also termed “stanolone”), 17-α methyl testosterone, 5α-dihydrotestosterone, 17α-methyl-5α-DHT, Fluoxymesterone (Halotestin®), Danazol (Danocrine®), dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone. The aforementioned testosterone esters are commercially available or may be readily prepared using techniques known to those skilled in the art. (Generally, the 17-hydroxyl group of the testosterone molecule is caused to react with a suitable organic acid under esterifying conditions, such conditions typically involving the use of a strong acid such as sulfuric acid, hydrochloric acid, or the like, and a temperature sufficient to allow the reaction to proceed at reflux.)

Nucleic Acid Agents

In some embodiments, an agent that increases a level and/or an activity of an androgen receptor is an agent that increases the level of AR mRNA and/or protein. Such agents include, but are not limited to, agents that increase the level of transcription of an AR gene, agents that increase stability of a AR mRNA, agents that reduce binding between an AR and apoE4, agents that increase the level of cytosolic and/or nuclear AR, and agents that increase the level of androgen-bound AR that enters the nucleus or retard the level of AR that leaves the nucleus.

Agents that increase the level of AR in the cytosol and/or nucleus include nucleic acids (also referred to herein as a “polynucleotide” or a “construct”) that include a nucleotide sequence that encodes an AR, where the nucleotide sequence is under transcriptional control of one or more control elements that regulate transcription and/or translation. Of particular interest are control elements that provide for higher levels of transcription in a cell in which AR is normally expressed, e.g., the control elements provide for at least 1.5 fold, at least 2 fold, at least 2.5 fold, at least 3 fold, or higher, levels of AR in the cytosol and/or nucleus compared to the level of AR found in the cytosol and/or nucleus in a neuronal cell, particularly when compared to the level of AR found in the cytosol and/or nucleus of a neuronal cell of an individual having one or two apoE4 alleles.

Nucleic acid “control sequences” or “regulatory elements” refer collectively to promoter sequences, ribosome binding sites, polyadenylation signals, transcription termination sequences, upstream regulatory domains, enhancers, and the like, which collectively provide for the transcription and translation of a coding sequence in a eukaryotic cell.

Suitable control elements include neuronal cell-specific promoters. Neuronal cell specific promoters and other control elements are well known in the art and include, but are not limited to, promoters and other control elements from the following genes: tyrosine hydroxylase (TH), dopamine beta hydroxylase (DBH), phenylethanolamine N-methyltransferase (PNMT), myelin basic protein (MBP), choline acetyltransferase (ChAT), glial fibrillary acidic protein (GFAP), neuron specific enolase (NSE), the neurofilament (NF) proteins (e.g., NF-L, NF-M, NF-H, and the like).

In many embodiments, the nucleic acid lacks a sequence that encodes a nuclear export signal. In these embodiments, the encoded AR is retained in the nucleus, and cognition is enhanced.

Nucleic acids that increase a level of AR in the cytosol and/or nucleus are generally provided in a vector. Vectors include, but are not limited to, plasmids; cosmids; viral vectors; artificial chromosomes (HACs, YACs, BACs, etc.); mini-chromosomes; and the like. Vectors are amply described in numerous publications well known to those in the art, including, e.g., Short Protocols in Molecular Biology, (1999) F. Ausubel, et al., eds., Wiley & Sons.

Other components may be included in the vector such as a detectable marker (e.g., a gene encoding a green fluorescent protein or a β-galactosidase-encoding gene), and/or a selectable marker (e.g., an antibiotic resistance gene, such as a neomycin resistance gene) to aid in selection and/or visualization of cells containing and/or expressing the construct, an origin of replication for stable replication of the construct in a bacterial cell (preferably, a high copy number origin of replication), a nuclear localization signal, or other elements which facilitate production of the expression construct, the protein encoded thereby, or both.

A nucleic acid can be associated with a viral delivery vehicle for delivery into a host. As used herein, a “viral delivery vehicle” intends that the polynucleotide to be delivered is encapsidated in a viral particle. Numerous viral genomes useful in in vivo transformation and gene therapy are known in the art, or can be readily constructed given the skill and knowledge in the art. Included are replication competent, replication deficient, and replication conditional viruses. Viral vectors include adenovirus, mumps virus, a retrovirus, adeno-associated virus, herpes simplex virus (HSV), cytomegalovirus (CMV), vaccinia virus, and poliovirus, and non-replicative mutants/variants of the foregoing. In some embodiments, a replication-deficient virus is capable of infecting slowly replicating and/or terminally differentiated cells, since the respiratory tract is primarily composed of these cell types. For example, adenovirus efficiently infects slowly replicating and/or terminally differentiated cells. In some embodiments, the viral genome itself, or a protein on the viral surface, is specific or substantially specific for cells of the targeted cell. A viral genome can be designed to be target cell-specific by inclusion of cell type-specific promoters and/or enhancers operably linked to a gene(s) essential for viral replication.

Where a replication-deficient virus is used as the viral genome, the production of virus particles containing either DNA or RNA corresponding to the polynucleotide of interest can be produced by introducing the viral construct into a recombinant cell line which provides the missing components essential for viral replication and/or production. Preferably, transformation of the recombinant cell line with the recombinant viral genome will not result in production of replication-competent viruses, e.g., by homologous recombination of the viral sequences of the recombinant cell line into the introduced viral genome. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992; Kawano et al., Virol., 179:857-861, 1990; Elango et al., J Gen. Virol. 69:2893-28900, 1988.

A nucleic acid can also be administered using a non-viral delivery vehicle. “Non-viral delivery vehicles” (“non-viral vectors”) include chemical formulations containing naked or condensed polynucleotides (e.g, a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel et al. 1992 Am. J. Respir. Cell Mol. Biol. 6:247-52)). Thus “non-viral delivery vehicle” includes vectors composed of polynucleotides plus viral particles where the viral particles do not contain the polynucleotide of interest. “Non-viral delivery vehicles” include bacterial plasmids, viral genomes or portions thereof, wherein the polynucleotide to be delivered is not encapsidated or contained within a viral particle, and constructs comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Felgner et al (U.S. Pat. Nos. 5,264,618 and 5,459,127; PNAS 84:7413-7417, 1987; Annals N.Y. Acad. Sci. 772:126-139, 1995); they may also consist of neutral or negatively charged phospholipids or mixtures thereof including artificial viral envelopes as disclosed by Schreier et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI; Boussif et al., PNAS 92:7297-7301, 1995) which molecules can also serve as condensing agents. These carriers may be dissolved, dispersed or suspended in a dispersion liquid such as water, ethanol, saline solutions and mixtures thereof. A wide variety of synthetic polymers are known in the art and can be used.

The nucleic acid is introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. Non-limiting examples of methods for transforming cells include the use of fusogenic lipid vesicles (liposomes incorporating cationic lipids such as lipofection; see Felgner et al., Proc. Natl. Acad. Sci. U.S.A. 84:7413-7417 (1987)); direct injection of DNA (Wolff, et al., Science (1990) 247:1465-1468); and pneumatic delivery of DNA-coated gold particles with a device referred to as the gene gun (Yang et al., Proc. Natl. Acad. Sci. U.S.A. 1990; 87:1568-9572). Morsy et al. reviews several of the different techniques useful in transformation of cells ex vivo or in vivo and provides citations of numerous publications in each area (Morsy et al., JAMA 270:2338-2345 (1993)). Oral delivery of DNA constructs can also be carried out. See, e.g., U.S. Pat. No. 6,258,789.

Whether the levels of AR mRNA are increased is readily determined using any known method. Suitable methods include a polymerase chain reaction (PCR) reaction; in situ hybridization; and RNA blot hybridization. The effectiveness of any given nucleic acid or other agent in increasing AR mRNA levels in an experimental system following gene therapy in vivo is determined using a PCR technique. For example, a nucleic acid as described above is introduced into a cell, e.g., a neuronal cell, in vitro, or a neuronal cell in vivo and, after a suitable time, a cDNA copy of mRNA from the cell is made. Using oligonucleotides of from about 15 nucleotides to about 50 nucleotides in length that serve as forward and reverse primers and that specifically prime synthesis of a cDNA copy of an AR mRNA, the level of AR mRNA is determined, and compared to a cell not transformed with the nucleic acid. The cDNA sequences of AR from various species are known. See, e.g., GenBank Accession Nos. NM_{—}012502, NM_{—}013476, and NM_{—}000044. Those skilled in the art, given the publicly available sequences of AR cDNA of various species, can readily design forward and reverse PCR primers that will amplify a cDNA corresponding to an AR mRNA.

Whether the levels of AR mRNA are increased in a cell is also readily determined using a Northern blot analysis technique. For example, mRNA is isolated from a neuronal cell that is transformed with a nucleic acid as described above, the mRNA is transferred to a membrane (e.g., nylon, polyvinyl pyrollidone, nitrocellulose, etc.), and the membrane is probed with a detectably labeled polynucleotide that hybridizes to AR mRNA.

Whether a given agent is effective in increasing cytosolic AR polypeptide levels is determined using any known method, e.g., an immunological assay (e.g., immunoprecipitation, Western blotting, enzyme linked immunosorbent assay, and the like); a functional assay (e.g., transcription of a reporter gene that is operably linked to a control element that responds to AR, and that is integrated into the genome); a ligand binding assay; and the like. A ligand binding assay can be carried out by determining binding of a detectably labeled AR ligand to cytosolic or nuclear AR. An example of a method for determining a level of AR in the cytosol is described in Example 3.

Agents that Reduce apoE Binding to AR

In some embodiments, an agent that increases a level and/or an activity of an androgen receptor is an agent that inhibits apoE binding to AR. Of particular interest are agents that inhibit a binding event between an apoE4 polypeptide and an AR, where the binding event occurs in the cytosol or nucleus. Suitable agents encompass numerous chemical classes, typically synthetic, semi-synthetic, or naturally-occurring inorganic or organic molecules. Suitable agents are small organic compounds having a molecular weight of more than 50 and less than about 2,500 daltons. Suitable agents may comprise functional groups necessary for structural interaction with proteins, particularly hydrogen bonding, and may include at least an amine, carbonyl, hydroxyl or carboxyl group, and may contain at least two of the foregoing functional chemical groups. An agent may comprise cyclical carbon or heterocyclic structures and/or aromatic or polyaromatic structures substituted with one or more of the above functional groups. Suitable agents are also found among biomolecules including peptides, saccharides, fatty acids, steroids, purines, pyrimidines, derivatives, structural analogs or combinations thereof.

Whether a given agent inhibits a binding event between apoE4 and an AR can be determined using any known method. For example, a binding assay can be carried out, in which either the apoE polypeptide or the AR is bound to a solid support. Binding between the apoE polypeptide and the AR is determined using any known assay, e.g., BRET (bioluminescence resonance energy transfer), FRET (fluorescence resonance energy transfer), ELISA (enzyme linked immunosorbent assay), a fluorescence quenching assay; a fluorescence anisotropy assay; an immunological assay; and an assay involving binding of a detectably labeled protein to an immobilized protein and the like. Such assays are well known in the art. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. To determine whether a given agent inhibits a binding event between an apoE polypeptide and an AR polypeptide, a given agent is added to an assay mixture that includes the apoE and the AR, and the effect, if any, of the agent on apoE/AR binding is determined using an assay that measures apoE/AR binding.

Whether a given agent inhibits binding between apoE4 and AR can also be determined using an assay involving contacting a cell with a test agent; and determining whether the test agent has an effect on apoE4 binding to AR, where the cell produces apoE4 and includes a construct that comprises an androgen response element operably linked to a reporter gene.

Pharmaceutical Formulations, Dosages, and Routes of Administration

An agent that increases the level and/or activity of an AR is generally administered to the host as a pharmaceutical composition that includes an effective amount of the agent in a pharmaceutically acceptable vehicle. In the subject methods, the active agent(s) may be administered to the host using any convenient means capable of resulting in the desired improvement on cognitive function. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, topical patches and aerosols.

As such, administration of the agents can be achieved in various ways, including oral, buccal, rectal, parenteral, intraperitoneal, intradermal, transdermal, intracheal, topical, etc., administration. In pharmaceutical dosage forms, the agents may be administered in the crystalline form or in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

Topical preparations are also of use. Topical formulations of androgenic compositions are known in the art. See e.g., U.S. Pat. Nos. 5,622,944; 5,635,203; 5,785,991 and 5,882,676; the disclosures of which are herein incorporated by reference.

The term “unit dosage,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Detecting an apoE Allele

In certain embodiments, the subject methods further include a step of evaluating whether the host carries an allele for the human ApoE4 isoform. A variety of different methods are known and currently available to those of skill in the art for detecting the presence of the human ApoE4 allele in a host. Representative methods are now described in greater detail.

The step of detecting the presence or absence of ApoE4 or of DNA encoding such isoform (including the number of alleles for ApoE4) may be carried out either directly or indirectly by any suitable means. A variety of techniques are known to those skilled in the art. All generally involve the step of collecting a sample of biological material containing either DNA or ApoE from the subject, and then detecting whether or not the subject possesses ApoE4 or DNA encoding such isoform from that sample. For example, the detecting step may be carried out by collecting an ApoE sample from the subject (for example, from cerebrospinal fluid, plasma, or any other fluid or tissue containing ApoE), and then determining the presence or absence of an ApoE4 isoform in the ApoE sample (e.g., by-isoelectric focusing, western blot or dot blot analysis, or immunoassay).

The isolation and characterization of ApoE is described, for example, in Rall et al., Methods in Enzymology 128:273-287 (1986), Davignon et al., Arteriosclerosis 8:1-21 (1988), and in Warnick et al., Clin. Chem. 25:279-284 (1979), all of which are incorporated by reference herein. Isoelectric focusing is an electrophoretic technique by which the molecules are separated based on their isoelectric points (pI) along a continuous pH gradient. Reference proteins, commercially available (e.g., Sigma Chemical Company, St. Louis, Mo.), are used to indicate a gradient along which the sample proteins match up according to where their pH matches their pI. In Warnick, et al. very-low-density apolipoproteins are isolated from plasma samples and applied to isoelectric focusing gels and the isoelectric focusing patterns of the ApoE isoforms are obtained. According to the Warnick et al. procedure, pI values of the ApoE isoform E4 was about 6.1 respectively in 8M urea at 4° C. See also, Pagnan et al., J. Lipid. Res. 18:613-622 (1977) and Utermann et al., FEBS Lett. 56:352-355 (1975). Various isoelectric focusing-type techniques are also provided in Rall et al. supra, including analytical isoelectric focusing, cysteamine treatment, neuraminidase treatment, sodium dodecyl sulfate-polyacrylamide gel electrophoresis, as well as amino acid analysis and nucleic acid sequence analysis, and capillary isoelectric focusing is described in H. Swartz, Bio/Technology 12, 408-09 (April 1994).

In the alternative, the detecting step may be carried out by collecting a biological sample containing DNA from the subject, and then determining the presence or absence of DNA encoding an ApoE4 isoform in the biological sample. Any biological sample which contains the DNA of that subject may be employed, including tissue samples and blood samples, with blood cells being a particularly convenient source. The amino acid sequences and nucleic acid sequences ApoE4 are known and described. See, for example, Paik et al., Proc. Nat'l Acad. Sci. U.S.A. 82:3445-3449 (1985) for the nucleic acid sequence Mahley, Science, 240:622-630 (1988) for the amino acid sequence information.

Determining the presence or absence of DNA encoding an ApoE4 isoform may be carried out with an oligonucleotide probe labeled with a suitable detectable group, or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the gene encoding an ApoE4 isoform. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. patent references cited herein be incorporated herein by reference).

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. U.S.A. 89, 392-396 (1992); G. Walker et al., Nucleic Acids Res. 20, 1691-1696 (1992)), transcription-based amplification (see D. Kwoh et al., Proc. Natl. Acad Sci. U.S.A. 86, 1173-1177 (1989)), self-sustained sequence replication (or “3SR”) (see J. Guatelli et al., Proc. Natl. Acad. Sci. U.S.A. 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred. DNA amplification techniques such as the foregoing can involve the use of a probe, a pair of probes, or two pairs of probes which specifically bind to DNA encoding ApoE4, but do not bind to DNA encoding ApoE2 or ApoE3 under the same hybridization conditions, and which serve as the primer or primers for the amplification of the ApoE4 DNA or a portion thereof in the amplification.

In general, an oligonucleotide probe which is used to detect DNA encoding ApoE4 is an oligonucleotide probe which binds to DNA encoding ApoE4, but does not bind to DNA encoding ApoE2 or ApoE3 under the same hybridization conditions. The oligonucleotide probe is labeled with a suitable detectable group, such as those set forth below in connection with antibodies.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all ApoE allelic types, the types can be distinguished by hybridization with allelic specific probe, by restriction endonuclease digestion, by electrophoresis on denaturing gradient gels, or other techniques. A PCR protocol for determining the ApoE genotype is described in Wenham et al., The Lancet 337:1158-1159 (1991), incorporated by reference herein. Examples of primers effective for amplification and identification of the ApoE isoforms are described therein. Primers specific for the ApoE polymorphic region (whether ApoE4, E3 or E2) can be employed. In Wenham, for example, PCR primers are employed which amplify a 227 bp region of DNA that spans the ApoE polymorphic sites (codons 112 and 158, which contain nucleotides 3745 and 3883). The amplified fragments are then subjected to restriction endonuclease CfoI which provides different restriction fragments from the six possible ApoE genotypes which may be recognizable on an electrophoresis gel. See also, Hixon et al., J. Lipid Res. 31:545-48 (1990); Houlston et al., Hum. Genet. 83:364-365 (1989) Wenham et al., Clin. Chem. 37:241-244 (1991); and Konrula et al., 36:2087-92 (1990) for additional methods, all of which are incorporated by reference herein.

Ligase chain reaction (LCR) is also carried out in accordance with known techniques. See, e.g., R. Weiss, Science 254, 1292 (1991). In general, the reaction is carried out with two pairs of oligonucleotide probes: one pair binds to one strand of the sequence to be detected; the other pair binds to the other strand of the sequence to be detected. Each pair together completely encompasses the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

As an alternative to isoelectric focusing and techniques for allele detection, the step of determining the presence or absence of the ApoE4 isoform in a sample may be carried out by an antibody assay with an antibody which selectively binds to ApoE4 (i.e., an antibody which binds to ApoE4 but exhibits essentially no binding to ApoE2 or ApoE3 in the same binding conditions). When one wishes to determine the precise ApoE complement of a patient and whether or not that patient is homozygous or heterozygous for ApoE4, then antibodies which selectively bind to ApoE2 and ApoE3 may also be employed (i.e., an antibody which binds to ApoE2 but exhibits essentially no binding to ApoE3 or ApoE4 in the same binding conditions; an antibody which binds to ApoE3 but exhibits essentially no binding to ApoE2 or ApoE4 in the same binding conditions). Antibodies used to selectively or specifically bind ApoE2, ApoE3, and ApoE4 can be produced by any suitable technique. For example, monoclonal antibodies may be produced in a hybridoma cell line according to the techniques of Kohler and Milstein, Nature 265, 495-97 (1975). ApoE2, ApoE3, or ApoE4 may be obtained from a human patient determined to be homozygous therefore, then purified by the technique described in S. Rallet al., Methods in Enzymol. 128, 273 (1986), and used as the immunogen for the production of monoclonal or polyclonal antibodies. Purified ApoE isoforms may be produced by recombinant means to express a biologically active isoform, or even an immunogenic fragment thereof may be used as an immunogen. Monoclonal Fab fragments may be produced in Escherichia coli from the known sequences by recombinant techniques known to those skilled in the art. See, e.g., W. Huse, Science 246, 1275-81 (1989) (recombinant Fab techniques); P. Wenham et al., Lancet 337, 1158 (1991) (ApoE PCR primers). The DNA encoding one subtype of ApoE can be obtained and converted to the other by site-directed mutagenesis. See, e.g., T. Kunkel et al., Methods in Enzymol. 154, 367-382 (1987); T. Kunkel, U.S. Pat. No. 4,873,192.

The term “antibodies” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies, and include antibody fragments such as, for example, Fab, F(ab′)₂, and Fv fragments, and the corresponding fragments obtained from antibodies other than IgG. For this invention, an antibody selectively or specifically binding ApoE or a particular ApoE isoform (ligand) generally refers to a molecule capable of reacting with or otherwise recognizing or binding such a ligand. An antibody has binding affinity for a ligand or is specific for a ligand if the antibody binds or is capable of binding the ligand as measured or determined by standard antibody-antigen or ligand-receptor assays, for example, competitive assays, saturation assays, or standard immunoassays such as ELISA or RIA. This definition of specificity applies to single heavy and/or light chains, CDRs, fusion proteins or fragments of heavy and/or light chains, that are specific for the ligand if they bind the ligand alone or in combination.

Antibody assays (immunoassays) may, in general, be homogeneous assays or heterogeneous assays. In a homogeneous assay the immunological reaction usually involves the specific antibody, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels which may be employed include free radicals, radioisotopes, fluorescent dyes, enzymes, bacteriophages, coenzymes, and so forth.

In a heterogeneous assay approach, the reagents are usually the specimen, the antibody of the invention and a system or means for producing a detectable signal. Similar specimens as described above may be used. The antibody is generally immobilized on a support, such as a bead, plate or Slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al., U.S. Pat. No. 4,376,110 to David et al., U.S. Pat. No. 4,275,149 to Litman et al., U.S. Pat. No. 4,233,402 to Maggio et al., and U.S. Pat. No. 4,230,767 to Boguslaski et al. Antibodies which selectively bind an ApoE isoform (i.e., bind to one of ApoE2, ApoE3 or ApoE4 while showing essentially no binding to the other under the same binding conditions) may be conjugated to a solid support suitable for a diagnostic assay (e.g., beads, plates, slides or wells formed from materials such as latex or polystyrene) in accordance with known techniques, such as precipitation. Antibodies which bind an ApoE isoform may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

In certain embodiments, a kit for detecting for the presence or absence of ApoE4 is employed, where the kit may include one or more reagents for carrying out one or more of the above described ApoE4 isoform, e.g., a nucleic acid for detection of the ApoE4 gene, an ApoE4 specific antibody, etc.

Methods and kits for use in detecting the presence of the ApoE4 isoform are further described in U.S. Pat. No. 5,508,167, the disclosure of which is herein incorporated by reference.

Where the subject methods include a step of detecting whether the subject carries the ApoE4 allele, in certain embodiments this step typically occurs before administration of the androgenic agent, such that the appropriateness of androgenic therapy is determined prior to administration of the androgen agent. In other words, the subject is first screened for the presence of the ApoE4 allele. If the screen is positive, androgenic therapy is determined to be appropriate. If the screen is negative, androgenic therapy is determined to be not appropriate and is not commenced. In this step, the subject may be determined to be heterozygous or homozygous for the ApoE4 allele.

Utility

The subject methods find use in any application where the improvement of a cognitive function is desired. The subject methods find use in the treatment of cognitive conditions in a variety of different hosts. As such, the subject methods find use in the treatment of female hosts, as described above, suffering from a cognitive decline disease condition. The subject methods also find use in the treatment of ApoE4 allele containing hosts, e.g., male or female hosts.

By cognitive decline disease condition is meant a disease condition which is characterized by a deterioration or worsening of at least one cognitive function, such as a deterioration in memory, a deterioration in learning ability, etc. One particular class of cognitive decline diseases which may be treated according to the subject methods are neurodegenerative disease conditions, and more particularly adult onset neurodegenerative disease conditions, such as Alzheimer's disease.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as deterioration in memory or learning ability or other cognitive function. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

As mentioned above, in these applications an effective amount of androgenic agent is administered to the host. By “effective amount” is meant a dosage sufficient to produce a desired result, where the desired result is generally an amelioration or alleviation, if not complete cessation, of one or more symptoms of the neurodegenerative disease being treated, particularly the cognitive decline symptoms, e.g., memory decline, learning ability decline, and the like.

The subject methods also find use in the treatment of apoE4-associated disorders. As used herein, an “apoE4-associated disorder” is any disorder that is caused by the presence of apoE4 in a cell, in the serum, in the interstitial fluid, in the cerebrospinal fluid, or in any other bodily fluid of an individual; any physiological process or metabolic event that is influenced by apoE4 domain interaction; any disorder that is characterized by the presence of apoE4; a symptom of a disorder that is caused by the presence of apoE4 in a cell or in a bodily fluid; a phenomenon associated with a disorder caused by the presence in a cell or in a bodily fluid of apoE4; and the sequelae of any disorder that is caused by the presence of apoE4. ApoE4-associated disorders include apoE4-associated neurological disorders and disorders related to high serum lipid levels. ApoE4-associated neurological disorders include, but are not limited to, sporadic Alzheimer's disease; familial Alzheimer's disease; poor outcome following a stroke; poor outcome following traumatic head injury; and cerebral ischemia. Phenomena associated with apoE4-associated neurological disorders include, but are not limited to, neurofibrillary tangles; amyloid deposits; memory loss; and a reduction in cognitive function, as described in greater detail above. ApoE4-related disorders associated with high serum lipid levels include, but are not limited to, atherosclerosis, and coronary artery disease. Phenomena associated with such apoE4-associated disorders include high serum cholesterol levels.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In addition to the above methods of treatment, the subject methods also find use in the prophylactic or preventative treatment regimens. In such methods, the host is administered an amount of an androgenic agent, typically according to a dosage schedule (.e.g., daily, weekly, monthly etc.), that is sufficient to prevent the occurrence of at least symptoms of the ApoE4 associated disorder, e.g., decline in cognition. Thus, the host may first be identified as being at risk for the disorder, e.g., cognitive decline condition, for example by testing for the presence of ApoE4 allele. Following identification of being at risk, the host is commenced on androgenic therapy to prevent or at least slow the occurrence of the disorder or symptoms associated therewith.

Kits

Kits with unit doses of an active androgenic agent, usually in slow-release devices (e.g., either patches or, alternatively, implants under the skin), oral or injectable doses, are provided. In such kits, in addition to the containers containing the unit doses will be an informational package insert describing the use and attendant benefits of the drugs in treating cognitive decline condition of interest and/or improving cognitive function. Preferred compounds and unit doses are those described herein above. In certain kits, also provided are means for detecting the presence of the ApoE4 isoform in a host.

The following examples are offered by way of illustration and not by way of limitation.

### EXPERIMENTAL

### Example 1

Androgens Improve Cognition in apoE4 Female Mice.

We tested whether administration of androgens might be successful for improving cognition in apoE4 female mice. (As described in Raber et al., Proc Natl Acad Sci USA Sep. 1, 1998;95(18):10914-9, where the female ApoE4 mice show age dependent spatial memory impairments that are detected from 6 months of age onward. See also U.S. Pat. No. 6,175,057, the disclosure of which is herein incorporated by reference).

Six-month-old female apoe^{-/-} mice expressing apoE3 or apoE4 in the brain at comparable levels, as described above, were anesthetized, and Silastic capsules filled with androgens (testosterone or dihydrotestosterone) were implanted subcutaneously; controls received placebo capsules (n=5-11 mice/genotype and treatment). More specifically, Silastic capsules (ID 1.57 mm; OD 3.18 mm, Dow Coming) were filled with testosterone or dihydrotestosterone (Sigma); placebo capsules were empty. Under methoxyflurane or isoflurane anesthesia, the hair in the back of the neck of mice was cleaned with ethanol, a 0.5-cm incision was made, a hormone or placebo capsule was implanted subcutaneously, and the incision was closed with sutures.

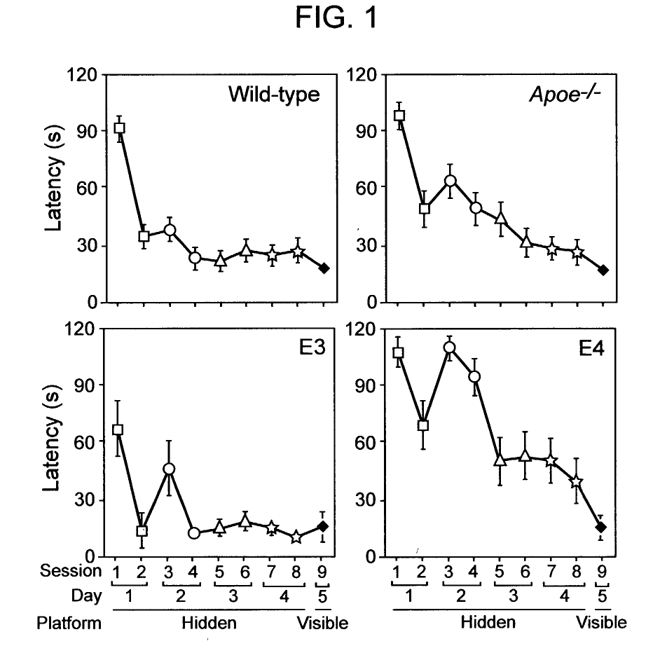

Testosterone and dihydrotestosterone exert androgenic effects by interacting with androgen receptors. Couse, J. Mol. Med. (1998) 76:497-511. In contrast to testosterone, dihydrotestosterone cannot be converted to 17-β estradiol by aromatase. Thus, comparing the effects of testosterone and dihydrotestosterone allows for a differentiation of androgen and estrogen effects. Eight days after implantation of the capsules, the mice were tested in the water maze. Testosterone improved learning and both testosterone and dihydrotestosterone improved memory retention in female apoE4 mice (FIG. 1). While all three groups learned to locate the hidden platform, there was a significant difference between the learning curves of testosterone- and placebo-treated mice during the hidden platform sessions (P<0.05 by repeated measures ANOVA). In the probe trial (platform removed), both testosterone- and dihydrotestosterone-treated, but not placebo-treated, apoE4 mice spent significantly more time searching in the target quadrant than in any of the other quadrants (* P<0.05, Tukey-Kramer test). Testosterone did not improve learning or spatial memory retention in female apoE3 mice. After the behavioral testing, plasma hormone levels were determined by radioimmunoassay (RIA) to ensure the effectiveness of the implants. Plasma hormone levels were measured by RIA (testosterone: ICN, Costa Mesa, Calif., and dihydrotestosterone: Diagnostic Systems Laboratories, Webster, Tex.) per manufacturer's instructions. Plasma testosterone levels in testosterone-treated female mice (5.61±0.74 ng/ml) were similar to those in untreated male mice and in men. These results indicate that brief androgen treatments dramatically improves the spatial learning and memory impairments in adult NSE-apoE4 female mice.

To determine whether apoE4 exerts gender-dependent detrimental effects also on nonspatial learning and memory, we assessed 6-month-old male and female apoE4, apoE3, wildtype, and apoe^{-/-} mice in a novel object recognition test. During the training session, mice were allowed to explore for 15 min an open field containing two objects. For the retention session (24 hours later), they were placed back into the same open field for 15 min, after one of the familiar objects was replaced with a novel object and the other familiar object with an exact replica. The percentage of time the mice spent exploring the novel versus the familiar object relative to the total amount of time they explored either object in the retention session was used as a measure of object recognition memory. In the training session, all groups of mice spent a comparable amount of time exploring each object. In the retention session, only female apoE4 mice showed significant deficits, whereas male apoE4 mice, and male and female apoE3, wildtype, and apoe^{-/-} mice had intact object recognition memory.

These results demonstrate that apoE4 induces deficits not only in spatial but also in nonspatial hippocampus- and cortex-dependent learning and memory. The resistance of male apoE4 mice against deficits in object recognition memory indicates that AR-dependent pathways protect against apoE4-induced cognitive impairments. The effects of androgens and AR receptor-dependent pathways on apoE4-induced cognitive deficits in our experimental models explain recent clinical observations in humans. Estrogen failed to preserve cognitive function in women with apoE4 (Yaffe, Neurology (2000) 54:1949-1954). In addition, testosterone, but not estrogen, levels in serum correlated positively with cognitive performance in older women (Barrett-Connor, JAMA (1999) 47:1289-1293) and testosterone therapy improved cognition in surgically menopausal women. Sherwin, Psychoneuroendocrinology (1988) 13: 345-357.

### Example 2

The Role of Androgen Receptors in Spatial and Nonspatial Learning and Memory

Based on our data demonstrating an important role for androgen receptors (ARs) in the gender-dependent cognitive impairments in apoE4 mice, we started to evaluate the role of ARs in cognition using mutant mice with a naturally occurring defect in the AR gene (testicular feminization mutant or tfm) on the C57/BL6J background. Because the trait in tfm mice is X-linked, males are androgen insensitive and females are partially insensitive. The hypothesis we are testing is that if ARs are important for spatial learning and memory, female tfm carrier mice will perform better than tfm male mice on such tests.

To assess spatial learning and memory, 6-8-month-old mice were tested in the water maze. A pool (diameter 140 cm) was filled with opaque water (24° C.) and mice were first trained to locate a visible platform (days 1-3) and then a submerged hidden platform (days 4-7) in two daily sessions 3.5 h apart, each consisting of three 60-sec trials (10-min inter-trial intervals). Mice that failed to find the hidden platform within 60 sec were put on it for 15 sec. For analysis of data, the pool was divided into four quadrants. During the visible platform training, the platform was moved to a different quadrant for each session. During the hidden platform training, the platform location was kept constant for each mouse (in the center of the target quadrant). The starting point at which the mouse was placed into the water was changed for each trial. Time to reach the platform (latency), path length, and swim speed were recorded with a Noldus Instruments EthoVision video tracking system set to analyze two samples per second. Since there were significant differences in average swim speeds between the groups (tfm females: 9.6±0.3 cm/sec, n=12; tfm males: 8.0±0.3 cm/sec, n=12,p<0.05 (Tukey-Kramer) during the visible platform sessions, the distance moved to locate the platform (cm) was used as the main measure for analysis. The distance moved values in all trials in which the mice did not find the platform were selected; an average distance moved for these trials was calculated to normalize and used for all trials in which the platform was not located. A 60-sec probe trial (platform removed) was carried out 1 hour after the last hidden platform session.

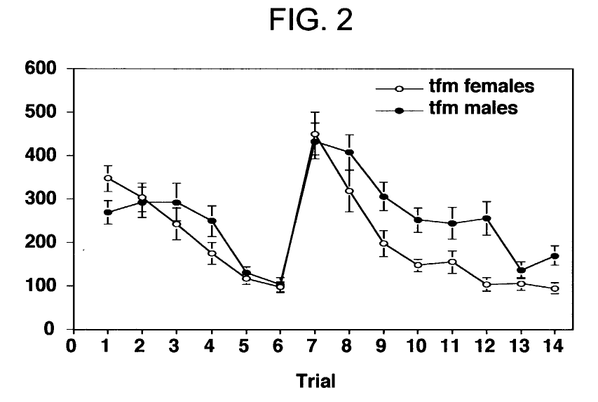

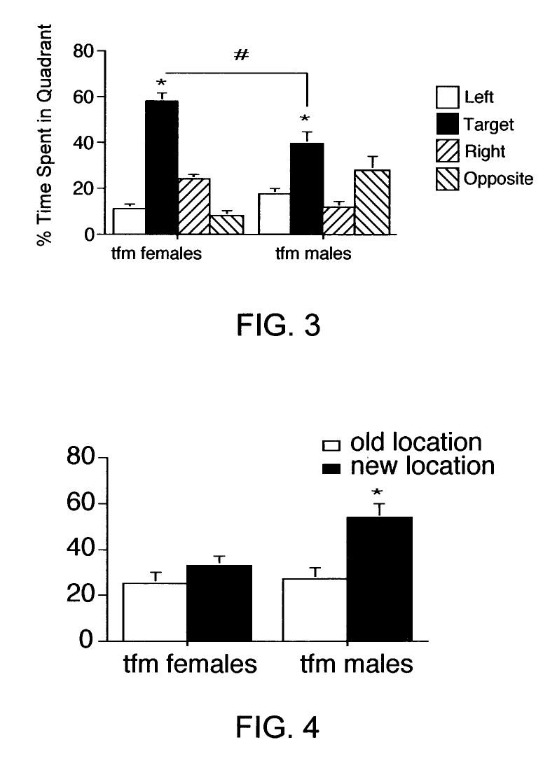

The tfm female and male mice significantly improved their performance over the visible and hidden platform sessions (FIG. 2,p<0.01). While there was no significant difference in the ability of the tfm female and male mice in locating the visible platform location, tfm female mice were significantly better than tfm male mice in locating the hidden platform location (p<0.01 (Tukey-Kramer). Both groups of mice swam continuously and in similar patterns, and there were no significant periods of passive floating. In the probe trial, both groups spent significantly more time in the target quadrant than in any of the other quadrants (p<0.01 (Tukey-Kramer) but tfm female mice spent significantly more time in the target quadrant than tfm male mice (p<0.05 (Tukey-Kramer) (FIG. 3). FIG. 3: *p<0.01 vs any other quadrant (Tukey-Kramer); #p<0.05 (Tukey-Kramer).

Rotorod Performance

To exclude that potential differences in body weights and/or sensorimotor function contributed to the differences in water maze performance, the mice were tested for rotorod performance over 5 trials. The tfm female and male mice significantly improved their rotorod performance with training (p<0.01) but there was no group difference in their performance.

Novel Location and Novel Object Recognition

Novel location and novel object recognition was used to evaluate whether androgen receptor deficiency has effects on nonspatial learning and memory (Malleret et al. (2001) Cell 104:675). After habituation to an open field, the mice were first trained in three consecutive trials and than tested in two consecutive trials with a 5-min inter-trial interval. For both the training and testing sessions, three objects were placed in the open field, and the animal was allowed to explore for 5 min. All objects were only used once and replicas were used in subsequent trials. Five min after the training trials, the animals were tested for recognition of the novel location of one of the familiar objects. In response to the change in location of one of the familiar objects, there was a significant difference between the tfm female and male mice in recognition of the novel location (p<0.05 (Tukey-Kramer); the tfm males, but not females, re-explored the object in the novel location more than they explored the object in the old location (p<0.01). Five min after the novel location test, the animals were tested for novel object recognition. The percentage of time the mice spent exploring the novel versus the familiar objects relative to the total amount of time they explored either object in this trial was used as a measure of object recognition memory. The tfm female and male mice spent a greater percentage of time exploring the novel object than the familiar objects relative to the total amount of time they explored all objects in this trial, but there was no group difference (tfm females: 59±9%, n=11; tfm males: 63±5%, n=8). The data are summarized in FIG. 4.

These data show that tfm females and males are not impaired in novel object recognition. In addition, these data demonstrate that tfm females, but not males, require sufficient androgen receptors for novel location recognition.

Elevated Plus Maze

When anxiety levels were assessed in the elevated plus maze, tfm female and male traveled similar distances (tfm females: 172±37 cm, n=11; tfm males: 122±29 cm, n=13), spent similar amounts of time (tfm females: 29±6 sec, n=11; tfm males: 22±4 sec, n=13), rested similar amount of time (tfm females: 13±2 sec, n=11; tfm males: 9±2 sec, n=13), and extended similar number of times over the edges (tfm females: 5.2±0.9 times, n=11; tfm males: 4.1±0.8 times, n=13) of the open arms of the plus maze. Thus, there were no differences in anxiety levels in the groups.

Open Field Activity

We measured active times and path lengths of the mice in a novel open area in the open field test. No significant differences were detected in active times (tfm females: 437±13 sec, n=12; tfm males: 430±21 sec, n=13), path lengths (wild-types: 3790±228 cm, n=12; tfm males: 3670±265 cm, n=13), and rearing events (tfm females: 86±9 sec, n=12; tfm males: 71±5 sec, n=13). Thus, there were no differences in overall activity levels in the groups.

### Example 3

Levels of Cytosolic AR

Materials and Methods

Cytosolic Androgen Receptor Binding Assay

For the determination of cytosolic AR binding, the method of McGinnis et al. ((1983) Brain Res. 275:75-82) was used, with slight modifications. Briefly, the entire neocortex and the hippocampal formation were dissected and homogenized separately with a glass-Teflon homogenizer (the pestle was raised and lowered 20 times) in 4 and 1 ml of TEDGM (10 mM Tris, 1.5 mM EDTA, 1 mM dithiothreitol, 10% glycerol, and 25 mM sodium molybdate), respectively. All steps were performed at 4° C. The homogenates were centrifuged at 1000×g for 10 minutes with an IEC Centra GB8R centrifuge. The supernatant was then centrifuged at 120,000×g for 30 minutes with a Beckman L8-70 Ultracentrifuge with a NVT65 rotor (Beckman Instruments, Fullerton, Calif.). Subsequently, 100 μl of the cytosolic fraction was incubated with different concentrations of ³H-R1881 (75.2 Ci/mmol; NEN, Boston, Mass.) and 10 μM triamcinolone acetonide in a total volume of 250 μl of TEDGM. Nonspecific binding was determined in separate incubation tubes containing the radioligand in the presence of a 100-fold excess of DHT. After overnight incubation, 100 μl of each sample was passed through a Sephadex LH-20 column to separate bound from free ³H-R1881. Columns were prepared with LH-20 that had been incubated in TEGM (10 mM Tris, 1.5 mM EDTA, 10% glycerol, and 25 mM sodium molybdate) for at least 24 hours before use. The swollen LH-20 was poured into blue pipette tips (Pipetman) fitted with 4 mm glass beads at the bottom. The columns were washed with 200 μl of TEDGM 15 minutes before use. Application of the sample was followed by 100 μl of TEDGM, and 15 minutes later by 200 μl of TEGM. The samples were eluted with 200 μl of TEGM, collected in scintillation vials, mixed with 7 ml of Ecoscint H (National Diagnostics, Atlanta, Ga.), and counted in a Beckman Coulter LS-6500 Multi-Purpose Scintillation Counter (Beckman Instruments). Results were expressed as femtomoles of ³H-R1881 bound per milligram of protein. The Bₘₐₓ was determined with GraphPad (San Diego, Calif.) Prism software (version 3.0 for Macintosh), suing a single curve-fit analysis. The protein concentrations were determined by the Bradford method.

Results

An AR binding assay to determine cytosolic AR levels in the neocortex and hippocampus of the apoE transgenic mice discussed in Example 1 was carried out. As depicted in FIGS. 6A-D, female and male apoE4 mice had lower cyosolic AR levels in the neocortex than apoE3, Apoe^{-/-}, or wild-type mice. Cytosolic AR levels in the hippocampus of apoE4 and apoE4 mice were not significantly different.

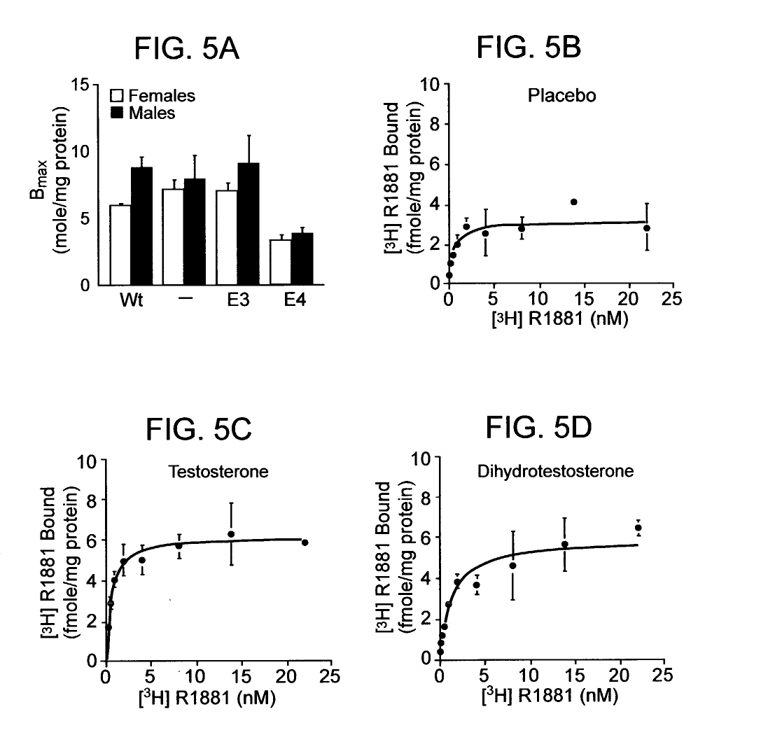

FIGS. 5A-D: Cyotosolic AR levels in the neocortex and response to androgen treatment are shown. Total cytosolic AR levels were determined from AR saturation curves using a single curve-fit analysis. Results are expressed as femtomoles of ³H-R1881 bound per milligram of protein. There were no differences in Kd among the groups. FIG. 5A: Untreated male and female NSE-apoE4 mice had lower cytosolic AR levels than untreated NSE-apoE3, Apoe^{-/-}, and wild-type (Wt) mice (n=3-7 mice per gender and genotype). FIGS. 5B-D: Effect of placebo (FIG. 5B), testosterone (FIG. 5C), and dihydrotestosterone (FIG. 5D) on AR saturation curves in female NSE-apoE4 mice (n=3-6 mice per treatment).

It is evident from the above results and discussion that improved methods for treating cognitive decline, particularly in female hosts, are provided. The subject methods provide an effective means for improving cognitive function, particularly in females suffering from cognitive decline disorders, e.g., neurodegenerative disease such as Alzheimer's Disease, etc. As such, the subject methods represent an important contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of improving a cognitive function of a female host, said method comprising:
administering to said female an effective amount of agent to improve said cognitive function, wherein said agent increases the level of biologically active androgen receptor in the cytosol or the nucleus of a cell.

2. The method of claim 1, wherein said agent is an androgenic agent.

3. The method according to claim 2, wherein said androgenic agent is a naturally occurring androgen.

4. The method according to claim 3, wherein said androgen is testosterone.

5. The method according to claim 2, wherein said androgenic agent is dihydrotestosterone or an analog thereof.

6. The method according to claim 1, wherein said agent disrupts binding between apoE4 and androgen receptor.

7. The method according to claim 1, wherein said agent is a nucleic acid that encodes androgen receptor.

8. The method according to claim 1, wherein said cognitive function is at least one of learning and memory.

9. The method according to claim 1, wherein said female host is a human.

10. The method according to claim 5, wherein said female human has an ApoE4 phenotype.

11. The method according to claim 1, wherein said method further comprises assaying said female host for the presence of at least one ApoE4 allele.

12. A method of treating a female host for a cognitive decline disease condition, said method comprising:
administering to said host an effective amount of an agent to treat said cognitive decline disease condition, wherein said agent increases the level of biologically active androgen receptor in the cytosol or nucleus of a cell.

13. The method according to claim 12, wherein said agent is an androgenic agent.

14. The method according to claim 13, wherein said androgenic agent is a naturally occurring androgen.

15. The method according to claim 14, wherein said androgen is testosterone.

16. The method according to claim 13, wherein said agent is dihydrotestosterone or an analog thereof.

17. The method according to claim 12, wherein said agent disrupts binding between apoE4 and androgen receptor.

18. The method according to claim 12, wherein said agent is a nucleic acid that encodes androgen receptor.

19. The method according to claim 12, wherein said cognitive function is at least one of learning and memory.

20. The method according to claim 12, wherein said female host is a human.

21. The method according to claim 20, wherein said female human has an ApoE4 phenotype.

22. The method according to claim 12, wherein said cognitive decline disease condition is a neurodegenerative disease condition.

23. The method according to claim 22, wherein said neurodegenerative disease condition is Alzheimer's disease.

24. The method according to claim 12, wherein said method further comprises assaying said female host for the presence of at least one ApoE4 allele.

25. A method of improving a cognitive function of a host having an ApoE4 allele, said method comprising:
administering to said ApoE4 allele containing host an effective amount of an agent to improve said cognitive function, wherein said agent increases the level of biologically active androgen receptor in the cytosol or nucleus of a cell.

26. The method according to claim 25, wherein said host suffers from a cognitive decline disease condition and said method is a method of treating said host for said cognitive decline disease condition.

27. The method according to claim 25, wherein said method further comprises assaying said host for the presence of said ApoE4 allele.

28. A method of treating a host having an ApoE4 associated disorder, said method comprising:
administering to said host an effective amount of an androgenic agent to treat said disorder.

29. The method according to claim 28, wherein said method further comprises assaying said host for the presence of an ApoE4 allele.

30. A kit for use in treating a female host for a cognitive decline disease condition, said kit comprising:
an androgenic agent in a pharmaceutically acceptable vehicle.

31. The kit according to claim 30, wherein said kit further comprises a means for detecting the presence of at least one ApoE4 allele in said female host.

32. The kit according to claim 30, wherein said androgenic agent is a naturally occurring androgen.

33. The kit according to claim 30, wherein said kit further comprises instructions for treating said cognitive decline disease condition.
